(19) United States
(12) Patent Application Publication
(10) Pub. No.: US 2003/0181962 A1
(43) Pub. Date: Sep. 25, 2003

(54) Low power energy therapy methods for bioinhibition

(76) Inventor: Jackson Streeter, Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/370,181

(22) Filed: Feb. 19, 2003

Related U.S. Application Data
(60) Provisional application No. 60/357,886, filed on Feb. 19, 2002.
(60) Provisional application No. 60/369,260, filed on Apr. 2, 2002.

(51) Int. Cl. A61N 5/067

(52) U.S. Cl. 607/89

(57) ABSTRACT

Energy therapy methods for inhibiting cell growth, differentiation, migration and proliferation are described, preferred methods including delivering to a subject in need thereof a bioinhibitory amount of electromagnetic energy having a wavelength in the visible to near-infrared wavelength range below about 820 nm, the bioinhibitory amount of electromagnetic energy being a predetermined power density (mW/cm²) of energy that is delivered to the subject. The methods will be useful in the treatment of cancers and inflammation.

### RELATED APPLICATION INFORMATION

This application claims priority under 35 U.S.C. §119(e) to U.S. Provisional Application Serial No. 60/357,886, filed Feb. 19, 2002 and U.S. Provisional Application Serial No. 60/369,260, filed Apr. 2, 2002, and also claims priority under 35 U.S.C. §120 to U.S. patent application Ser. No. 10/287,432, filed Nov. 1, 2002, the disclosures of which are hereby incorporated by reference in their entireties.

### BACKGROUND OF THE INVENTION

1. Field of the Invention

The invention relates in general to methods for the methods for the treatment of disease and conditions involving an excess of otherwise normal cellular activity including growth, differentiation, proliferation or migration, and more particularly to medical therapeutic methods using low power irradiation of body tissue with electromagnetic energy for inhibiting excess cellular activity.

2. Description of the Related Art

Certain mammalian diseases and conditions can be viewed as resulting from an excess of otherwise normal cellular activity. For example, cancer is frequently characterized as uncontrolled cellular growth, proliferation and migration. The normal inflammatory response to tissue injury includes the secretory and signaling activity of various cell types which, if uncontrolled or not adequately controlled, results in excessive and painful inflammation or excessive scar formation. Such cellular activity is supported by basic cellular functions such as respiration, protein synthesis, packaging, transport and metabolism, and the like.

Cancer is any type of malignant growth of body tissue. In contrast to normal cells, which reproduce and develop in controlled fashion under genetic controls, cancerous cells grow uncontrollably, fail to differentiate, and can propagate throughout the body from the site of origin to compete with and ultimately destroy or replace normal healthy cells in other parts of the body. Surgical excision of cancerous growths or tissue is known. However, surgery is not always a satisfactory or successful approach to cancer treatment. For example, surgery is not fully adequate to treat cancers that have metastasized, and surgery is not suitable for cancers characterized by abnormal tissue that is not highly localized, such as lymphomas. Chemotherapies and radiation therapy are not perfectly selective for cancer cells and can product severe side effects by destroying other rapidly proliferating cells yet normal healthy cells such as epithelial cells.

More specifically with regard to inflammation, when the body is injured, the normal response includes inflammation and formation of scar tissue. Usually the process of scar tissue formation is controlled, the tissue heals predictably and the injured area continues to function adequately with minimal or moderate scar presence. Rarely, the process of scar formation is not well controlled and excessive scar tissue is produced, either in the form of hypertrophic scars, or in the form of keloids in which scar tissue ultimately extends beyond the original wound margins.

The normal inflammatory response is initiated when injured tissue releases soluble messengers such as histamine that cause acute vasodilation of noninjured blood vessels in the vicinity of the injury, resulting in the classic reddening, heating, swelling and pain of inflammation. Other signaling molecules involved in inflammation and wound healing include plasma-derived bradykinins and prostaglandins, which contribute to changes in long-term changes in vascular permeability and vasodilation. Mast cells release hyaluronic acid and other proteoglycans into the would milieu, to bind with watery wound fluid to create a non-flowing gel. Myofibroblasts work to contract the would margins.

Wound healing culminates with the laying down of collagen that is synthesized and secreted by migratory fibroblasts that have traveled to the wound. In a complex process involving the formation of tropo-collagen molecules and coupling of the molecules to one another via intramolecular cross-links, collagenous filaments fill in the wound. A final step in wound healing is remodeling of the collagen that has been deposited, in which the process of collagen synthesis is balanced against a process of lysis of the collagen to reconfigure the scar tissue to approximate normal tissue. Collagen synthesis that is not well balanced by lysis produces abnormal scarring, including hypertrophic scars and keloids, which result from normal collagen synthesis combined with inhibited lysis. A known treatment for hypertrophic scarring includes applying pressure to the scar tissue to produce local ischemia that inhibits collagen synthesis.

While methods are known for treating hypertrophic scars, in which excessive scar formation is limited to the original wound margins, keloid formation is not well understood. Keloids are most commonly observed in certain areas of the body such as the earlobes, chest and shoulders, and certain people are more commonly afflicted than others, including redheads, young people of African or Afro-American descent, and elderly Caucasians. Known methods for treating keloid include injected steroids, intensive topical application of silicone gel sheeting, extending application of pressure through the use of pressure garments or pressure earrings (for earlobes), scar re-excision to re-initiate the healing process, and, only in especially intractable cases, short course radiation therapy.

In the medical surgical arts, high-energy laser radiation is now well accepted as a tool for cutting, cauterizing, and ablating biological tissue. High energy lasers of various wavelengths are now routinely used for vaporizing superficial skin lesions and, and to make deep cuts. For a laser to be suitable for use as a surgical laser, it must provide laser energy at a power sufficient to heat tissue to temperatures over 50 C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in biostimulatory effects while leaving tissue undamaged. For example, in rat models of myocardial infarction and ischemia-reperfusion injury, low energy laser irradiation reduces infarct size and left ventricular dilation, and enhances angiogenesis in the myocardium. (Yaakobis et al., J. Appl. Physiol. 90, 2411-19 (2001)). Low level laser therapy using low power lasers of less than 100 mW and at specific dosages of 1-10 Joules/cm² has been described for reducing inflammation, for treating joint pain, musculoskeletal injuries, and skin conditions, for promoting wound healing, and in dental applications.

Low level laser therapy has been described for easing the side effects of cancer patients who have undergone surgery, or have received radiation. Use of a low-energy He/Ne laser at a wavelength of 632.8 nm and power of 60 mW has been described for treating radiation-induced mucositis in patients with head and neck cancer. (Bensadoun et al., Support Care Cancer 7(4):244-52 (1999). Pre-surgical low level laser irradiation of patients with stage 1V stomach cancer produced increased T-active rosette cells and T-helper cells, while decreasing T-suppressor cells. (Mikhailov, et al., Laser & Technology. 7 (10:31-44 (1997)). However, low level laser energy has not been used in the treatment of cancer patients for limiting or preventing the cancer itself, but instead has been limited to treating the side effects of primary cancer therapy. Application of 890 nm laser energy at a minimal power may have a turmorstatic effect on experimental tumor cells. (Mikhailov, et al., Laser Therapy 4(4):169 (1991)). However, the application of low level laser energy to experimental tumor cells has been limited to the wavelength of 890 nm, using a laser energy source.

In addition, known low level laser therapy methods for treating disease or injury of any kind are limited in several respects. The known methods are limited to using the coherent light energy of a laser source, and at a specific wavelength selected for the particular tissue or application. Further, known low level laser energy methods are based on applying coherent laser energy at low power and at a specific dose or dosage range, i.e. an amount of laser energy measured in Joules/square centimeter. U.S. Pat. No. 5,464,436 describes treating injured tissue using a wavelength of 830 nm delivered from a laser energy source, using a very low power laser energy source of 5 mW to 70 mW, and using low dosages of 110 Joule/cm². Thus, known low level laser therapy methods are circumscribed by setting wavelength, power, laser energy dosage and time period of exposure to within specified limits. However, known low level laser therapy methods do not describe controlling other parameters that contribute to a photon density that is delivered to tissue and may play a key role in producing desirable photobiological effects on tissue.

Against this background, a high level of interest remains in finding new and improved therapeutic methods for the treatment of cancer and other diseases and conditions that involve an excess of cell growth, differentiation, proliferation and migration. In particular, a need remains for relatively inexpensive and non-invasive approaches to treating cancer and other such conditions that avoid the limitations of drug therapy and radiation.

### SUMMARY OF THE INVENTION

A method for inhibiting cell growth, differentiation, proliferation or migration in a subject in need of such treatment, is based primarily on the surprising finding that applying electromagnetic energy having a wavelength, in the visible to near-infrared wavelength range below about 820 nm, and particularly within the range of about 700 nm to about 800 nm, to biologic tissue appears, to have an inhibitory effect on cell growth, differentiation, proliferation or migration. In addition, the method is based on the finding that applying the electromagnetic energy at a pre-selected power density, i.e. controlling the power density at which the electromagnetic energy is applied, appears to be an important factor in producing the bioinhibitory effects.

Thus, a method for inhibiting cell growth, differentiation, proliferation or migration in a subject in need of such treatment comprises delivering a bioinhibitory amount of electromagnetic energy having a wavelength below about 820 nm in the visible to near-infrared wavelength range to the subject wherein delivering the bioinhibitory amount of electromagnetic energy comprises selecting a predetermined power density of the electromagnetic energy to deliver to the subject. In preferred embodiments, the predetermined power density is at least about 1 mW/cm² and may be selected from the range of about 1 mW/cm² to about 100 mW/cm². Lower and higher power densities capable of achieving desired results may also be used in accordance with the methods described herein.

Also provided is a method for inhibiting cell growth, differentiation, proliferation or migration in a subject in need of such treatment, wherein the method comprises delivering to the subject a bioinhibitory amount of electromagnetic energy having a wavelength in the visible to near-infrared wavelength range below about 820 nm wherein delivering the bioinhibitory amount of electromagnetic energy comprises controlling the power density of the electromagnetic energy to deliver to the subject. In preferred embodiments, controlling the power density of the electromagnetic energy includes selecting a dosage and power of an electromagnetic energy source that is sufficient to maintain the power density of the electromagnetic energy within a predetermined range of power density values while delivering the electromagnetic energy to the subject.

### BRIEF DESCRIPTION OF THE DRAWINGS

FIG. 1 is a perspective view of a first embodiment of a light therapy device; and

FIG. 2 is a block diagram of a control circuit for a light therapy device, such as is illustrated in FIG. 1.

### DETAILED DESCRIPTION OF THE PREFERRED EMBODIMENT

The low power energy therapy methods described herein may be practiced using, for example, a low level laser therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference in their entireties together with the references contained therein. Such apparatus, and other suitable apparatus, preferably include light energy sources, such as laser light sources, capable of emitting light energy having a wavelength in the visible to near-infrared wavelength range below about 820 rim. A handheld probe may be used for delivering the laser or light energy. The probe includes a laser source of light energy. The probe may include, for example, a single laser diode that provides about 100 mW to about 500 mW of total power output, or multiple laser diodes that together are capable of providing at least about 100 mW to about 500 mW of total power output. In preferred apparatus, the actual power output is variable using control unit electronically coupled to the probe, so that the power of the laser energy emitted can be adjusted in accordance with the required power density calculations as described infra.

The methods described herein preferably use electromagnetic energy having a wavelength in the visible to near-infrared wavelength range below about 820 nm. In one embodiment, the wavelength is in the range of about 700 nm to about 800 nm, a range of wavelengths that appears to be especially suitable for obtaining the desired bio-inhibitory effects on cellular growth, differentiation, proliferation or migration. In another embodiment, the wavelength is in the range of about 725 nm to about 785 rim. In one exemplary embodiment, the wavelength is about 730 nm, and in another exemplary embodiment, the wavelength is about 780 rim. Examples of suitable light sources for producing the electromagnetic energy include laser sources such as the semiconductor, continuously emitting GaAIAs laser (emitting at about 780 nm), and the crystalline pulsed lasers Alexandrite (emitting at 755 nm) and Ti:sapphire (emitting at 700-900 nm). Alternatively, the electromagnetic energy source is another type of diode, for example light-emitting diode (LED), or other light energy source, provided that the electromagnetic energy source has a wavelength in the visible to near-infrared wavelength range, below about 820 nm, preferably in the range of about 700 nm to about 800 nm, more preferably in the range from about 725 nm to about 785 nm, and most preferably about 730 nm or about 780 nm. The level of coherence of a light energy source is not critical. A light energy source used as the electromagnetic energy source need not provide light having the same level of coherence as the light provided by a laser energy source.

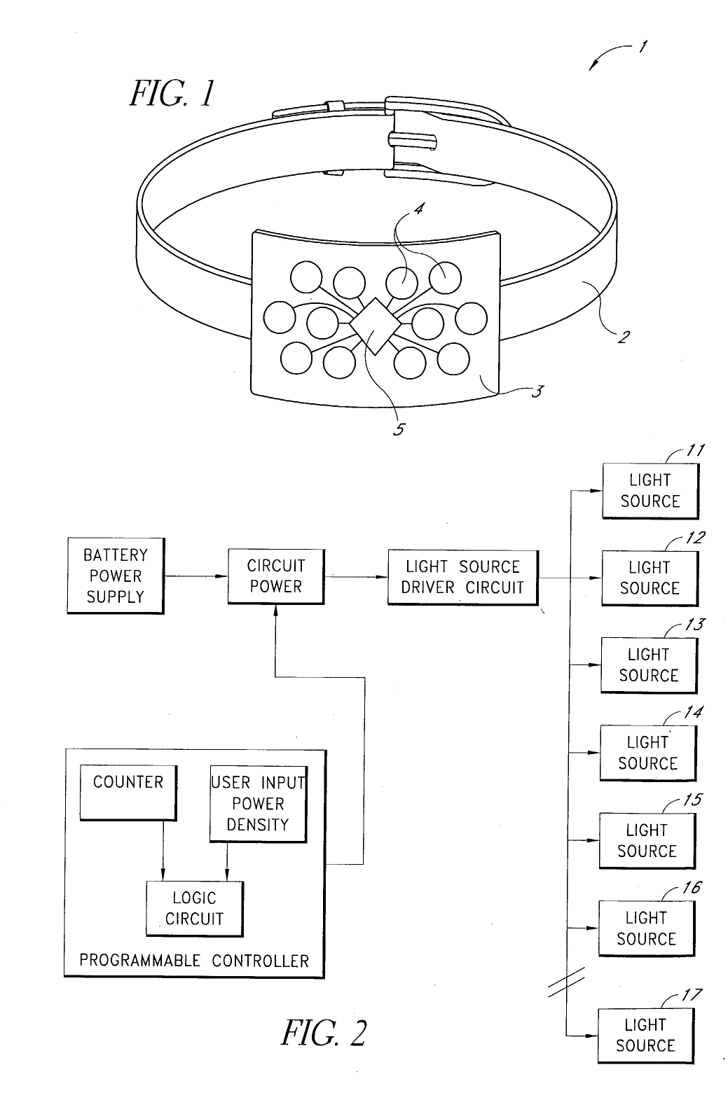

Another suitable light therapy apparatus is that illustrated in FIG. 1. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve the predetermined power density. The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly over a particular body part, such as a particular arm or leg joint, or around the chest or hips. The precise configuration of the strap is subject only to the limitation that the strap is capable of maintaining the light energy sources in a select position relative to the particular area of the body or tissue being treated. In an alternative embodiment, a strap is not used and instead the light source or sources are incorporated into or attachable onto a piece of fabric which fits securely over the target body portion thereby holding the light source or sources in proximity to the patient's body for treatment. The fabric used is preferably a stretchable fabric or mesh comprising materials such as Lycra or nylon. The light source or sources are preferably removably attached to the fabric so that they may be placed in the position needed for treatment.

In the exemplary embodiment illustrated in FIG. 1, a light therapy device includes a flexible strap and securing means such as mating Velcro strips configured to secure the device around the body of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are preferably secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body.

FIG. 2 is a block diagram of a control circuit according to one embodiment of the light therapy device. The programmable controller is configured to select a predetermined surface power density of the light energy sufficient to deliver a predetermined power density to the target area. The actual total power output if the light energy sources is variable using the programmable controller so that the power of the light energy emitted can be adjusted in accordance with what is calculated as being needed for treatment.

Examples of diseases and conditions involving an excess of cellular growth, differentiation, proliferation and migration and treated according to the present methods generally include cancers, cancer-related conditions and inflammation and inflammation-related diseases and conditions, especially those involving increased activity of fibroblasts and white blood cells, including diseases and conditions that involve the formation of scar tissue, including hypertrophic scars and keloids.

In addition, preferred methods are based, at least in part, on the finding that applying the electromagnetic energy at a pre-selected power density appears to be a decisive factor in producing the bioinhibitory effects. Thus, the method comprises delivering a bioinhibitory amount of electromagnetic energy having a wavelength below about 820 nm in the visible to near-infrared wavelength range to the subject wherein delivering the bioinhibitory amount of electromagnetic energy comprises selecting a predetermined power density of the electromagnetic energy to deliver to the subject. The predetermined power density is preferably at least about 1 mW/cm². In an exemplary embodiment, the power density is selected from the range of about 1 mW/cm² to about 100 mW/cm².

Preferred embodiments also include a method for inhibiting cell growth differentiation, proliferation or migration in a subject in need of such treatment, wherein the method comprises delivering to the subject a bioinhibitory amount of electromagnetic energy having a wavelength in the visible to near-infrared wavelength range below about 820 nm by controlling the power density of electromagnetic energy to deliver to the subject. Controlling the power density of the electromagnetic energy includes selecting a dosage and power of an electromagnetic energy source that is sufficient to maintain the power density of the electromagnetic energy within a predetermined range of power density values while delivering the electromagnetic energy to the subject.

However, the methods are based primarily on the surprising finding that select wavelengths of electromagnetic energy, applied at certain power densities to biologic tissue, appear to have bioinhibitory effects on the tissue. Without being bound by theory, it is believed that only electromagnetic energy having a wavelength in the visible to near-infrared wavelength range below about 820 nm provides a bioinhibitory effect on mitochondria that disrupts normal basic cellular functions such as respiration that are required to support cell growth and mitosis.

The term “bioinhibitory” refers to the process of disrupting normal basic cellular functions such as respiration and protein synthesis that support cell growth, differentiation, proliferation and migration. Additional basic cell functions that may be disrupted by application of electromagnetic energy having a wavelength in the visible to near-infrared wavelength range below about 820 nm, include, for example, protein transport and metabolism, intracellular and intercellular signaling, ion transport and electrolyte balance.

The terms “bioinhibitory” and “bioinhibitory effective” as used synonymously herein refer to a characteristic of an amount of electromagnetic energy at a particular wavelength. The amount of electromagnetic energy achieves the goal of disrupting normal basic cellular functions such as respiration and protein synthesis that support cell growth, differentiation, proliferation and migration, so that the activity of cells contributing to diseases and conditions such as cancer and inflammation are inhibited or prevented from carrying out their function, so that the activity of cells contributing to diseases and conditions such as a cancer and inflammation are inhibited or prevented from carrying out their function.

In preferred embodiments, treatment parameters include the following. Preferred power densities of light at the level of the target cells are at least about 1 mW/cm² and up to about 100 mW/cm², including about 5, 10, 15, 20, 30, 40, 50, 60, 70, 80, and 90 mW/cm² To achieve desired power densities, preferred light energy sources, or light energy sources in combination, are capable of emitting light energy having a total power output of at least about 1 mW to about 500 mW, including about 5, 10, 15, 20, 30, 50, 75, 100, 150, 200, 250, 300, and 400 mW, but may also be up to as high as about 1000 mW or below 1 mW.

The light source used in the light therapy is preferably a coherent source (i.e. a laser), and/or the light is substantially monochromatic (i.e. one wavelength or a very narrow band of wavelengths).

In some embodiments, the treatment proceeds continuously for a period of about 30 seconds to about 2 hours, more preferably for a period of about 1 to 20 minutes. The treatment may be terminated after one treatment period, or the treatment may be repeated with preferably about 1 to 2 days passing between treatments. The length of treatment time and frequency of treatment periods can be varied as needed to achieve the desired result.

During the treatment, the light energy may be continuously provided, or it may be pulsed. If the light is pulsed, the pulses are preferably at least about 10 ns long, including about 100 ns, 1 ms, 10 ms, and 100 ms, and occur at a frequency of up to about 1 kHz, including about 1 Hz, 10 Hz, 50 Hz, 100 Hz, 250 Hz, 500 Hz, and 750 Hz.

The low power energy therapy methods will be useful in treating or preventing cancer and inflammation or inflammation-related conditions, including side effects of inflammation, scar formation and excessive scarring. With respect to scar formation and excessive scarring, the low power energy therapy methods can be used to inhibit the activity of fibroblasts and white blood cells that contribute to the inflammation and scar formation processes.

Thus, a method for inhibiting cell growth, differentiation, proliferation or migration in a subject in need of such treatment involves delivering to the subject a bioinhibitory effective amount of electromagnetic energy having a wavelength in the visible to near-infrared wavelength range below about 820 nm, wherein delivering the bioinhibitory amount of electromagnetic energy comprises selecting a predetermined power density of the electromagnetic energy to deliver to the subject. A power density (mW/cm²) of the electromagnetic energy is selected and maintained within a specified range, by a controlling a dosage (Joules/cm²), time period (seconds or minutes) and power (mW) of an electromagnetic energy source such as a laser or light-emitting diode, sufficient to deliver type predetermined power density of energy to the subject.

Thus, delivering the bioinhibitory effective amount of electromagnetic energy includes selecting a dosage and power of the electromagnetic energy sufficient to deliver the predetermined power density of electromagnetic energy to the area of inflammation. The predetermined power density is preferably selected to be at least about 1 mW/cm². In one embodiment, the predetermined power density is selected from the range of about 1 mW/cm² to about 100 mW/cm², including about 2 mW/cm² to about 20 mW/cm². If the target tissue is below the surface of the skin, to deliver the predetermined power density at the level of the targeted tissue, a relatively greater surface power density of the energy is calculated taking into account attenuation of the energy as it travels through any intervening tissues including, for example, skin, muscle, and connective tissue. Factors known to affect penetration and to be taken into account in the power density calculation include skin pigmentation, and the location of the targeted tissue, particularly the depth of the tissue to be treated relative to the surface. For example, to obtain a desired power density of 100 mW/cm² in targeted cancerous brain tissue at a depth of 3 cm below the surface may require a surface power density of 400 mW/cm². The higher the level of skin pigmentation, the higher the required surface power density to deliver a predetermined power density of electromagnetic energy to a subsurface site.

Within the aforementioned preferred ranges, the precise power density selected for treating the patient may depend on a number of factors, including the specific wavelength of energy selected, the type or location of cancer or inflammation being treated, the clinical condition of the subject including the severity or stage of condition or disease being treated, and the like. Similarly, it should be understood that the power density of the energy may be adjusted to be combined with any other therapeutic agent or agents, especially pharmaceutical agents that together with the energy therapy achieve the desired biological effect of inhibiting cell functions in the targeted tissue. In combination with pharmaceutical agents, the selected power density for the energy therapy will again depend on a number of factors, including the specific energy wavelength chosen, the individual additional therapeutic agent or agents chosen, and the clinical condition of the subject.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for inhibiting cell growth, differentiation, proliferation or migration in a subject in need of such treatment, said method comprising delivering to the subject a bioinhibitory amount of electromagnetic energy having a wavelength in the visible to near-infrared wavelength range below about 820 nm wherein delivering the bioinhibitory amount of electromagnetic energy comprises selecting a predetermined power density of the electromagnetic energy to deliver to the subject.

2. A method in accordance with claim 1, wherein the predetermined power density is a power density of at least about 1 mW/cm².

3. A method in accordance with claim 1, wherein the predetermined power density is selected from the range of about 2 mW/cm² to about 20 mW/cm².

4. A method in accordance with claim 1, wherein the light energy has a wavelength of about 700 to about 800 nm.

5. A method in accordance with claim 4, wherein the light energy has a wavelength of about 730 nm to about 780 nm.

6. A method in accordance with claim 4, wherein the light energy has a wavelength of about 730 nm.

7. A method in accordance with claim 4, wherein the light energy has a wavelength of about 780 nm.

8. A method in accordance with claim 1, wherein delivering the bioinhibitory amount of electromagnetic energy comprises delivering the electromagnetic energy with a laser energy source.

9. A method in accordance with claim 1, wherein delivering the bioinhibitory amount of electromagnetic energy comprises delivering the electromagnetic energy with a non-coherent light energy source.

10. A method in accordance with claim 7, wherein delivering the bioinhibitory amount of electromagnetic energy with a non-coherent light energy source comprises delivering the electromagnetic energy with a light-emitting diode.

11. A method in accordance with claim 1, wherein the subject suffers from a cancer.

12. A method in accordance with claim 1, wherein the subject suffers from inflammation or an inflammation-related condition.

13. A method for inhibiting cell growth, differentiation, proliferation or migration in a subject in need of such treatment, said method comprising delivering to the subject a bioinhibitory amount of electromagnetic energy having a wavelength in the visible to near-infrared wavelength range below about 820 nm, wherein delivering the bioinhibitory amount of electromagnetic energy comprises controlling a power density of the electromagnetic energy to deliver to the subject.

14. A method in accordance with claim 13, wherein controlling the power density of the electromagnetic energy comprises selecting a dosage and power of an electromagnetic energy source sufficient to maintain the power density of the electromagnetic energy within a predetermined range of power density values while delivering the electromagnetic energy to the subject.

15. A method in accordance with claim 14, wherein selecting the dosage and power of the electromagnetic energy source sufficient to maintain the power density of the electromagnetic energy within a predetermined range of power density comprises selecting the dosage and power of the electromagnetic energy source sufficient to maintain the power density of the electromagnetic energy within a range of at least about 1 mW/cm² to about 100 mW/cm².

16. A method in accordance with claim 13, wherein delivering a bioinhibitory amount of electromagnetic energy having a wavelength in the visible to near-infrared wavelength range to the subject comprises delivering electromagnetic energy having a wavelength of about 700 nm to about 800 nm.

17. A method in accordance with claim 16, wherein delivering a bioinhibitory amount of electromagnetic energy having a wavelength in the visible near-infrared wavelength range to the subject comprises delivering electromagnetic energy having a wavelength of about 730 nm.

18. A method in accordance with claim 16, wherein delivering a bioinhibitory amount of electromagnetic energy having a wavelength in the visible to near-infrared wavelength range to the subject comprises delivering electromagnetic energy having a wavelength of about 780 nm.

19. A method in accordance with claim 13 wherein the subject suffers from a cancer.

20. A method in accordance with claim 13 wherein the subject suffers from inflammation or an inflammation-related condition.
